(19) United States
(12) Patent Application Publication
(10) Pub. No.: US 2026/0315371 A1
(43) Pub. Date: Oct. 15, 2026

(54) Milk-Derived Extracellular Vesicle Compositions and Methods of Production and Use.

(71) Applicant: Joseph William King, Leander, TX (US)

(72) Inventor: Joseph William King, Leander, TX (US)

(21) Appl. No.: 19/629,448

(22) Filed: Mar. 26, 2026

Related U.S. Application Data
(60) Provisional application No. 63/786,295, filed on Apr. 10, 2025.
(60) Provisional application No. 63/817,583, filed on Jun. 4, 2025.

(51) Int. Cl. A61K 8/98; A61K 8/14; A61K 8/44; A61K 8/67; A61K 8/73; A61K 9/19; A61K 9/50; A61K 35/20; A61Q 5/00; A61Q 19/00

(52) U.S. Cl. CPC A61K 8/986; A61K 8/14; A61K 8/44; A61K 8/675; A61K 8/735; A61K 9/19; A61K 9/5063; A61K 35/20; A61Q 5/00; A61Q 19/007

(57) ABSTRACT

A method of isolating extracellular vesicles from mammalian milk using filtration-based processing without required ultracentrifugation is disclosed. The method may include coarse and fine filtration, optional tangential flow filtration, temperature-controlled handling, stabilization with trehalose and milk-derived solids, and drying to produce stable powder or liquid compositions. The resulting extracellular vesicle compositions may be incorporated into topical, oral, and hybrid consumer products including cosmetic, personal care, and nutraceutical formulations. The invention further relates to scalable processes that preserve vesicle structure and associated molecular cargo, including microRNAs, while enabling formulation-ready mammalian milk-derived extracellular vesicle products.

### FIELD OF THE INVENTION

The present invention relates generally to extracellular vesicles derived from mammalian milk, including exosomes and related vesicular structures. More particularly, the invention relates to practical and scalable methods for isolating, concentrating, stabilizing, drying, storing, and formulating such vesicles and compositions containing such vesicles for cosmetic, nutraceutical, therapeutic, and related uses.

In representative embodiments, the invention addresses a manufacturing bottleneck in the commercialization of mammalian milk-derived extracellular vesicle products by replacing or reducing dependence on ultracentrifugation with staged filtration and optional tangential flow filtration. The invention also addresses downstream stabilization and formulation challenges associated with preserving vesicle integrity during drying, storage, reconstitution, and product incorporation.

### BACKGROUND OF THE INVENTION

Extracellular vesicles (EVs), including exosomes, are nanoscale lipid bilayer particles released by cells and biological fluids. These vesicles may carry proteins, lipids, nucleic acids, and microRNAs and are increasingly recognized as biologically active materials of interest for skin care, regenerative support, and oral wellness formulations. Milk is a particularly attractive source because it is naturally enriched in vesicular structures and may be obtained in commercially meaningful volumes.

Despite this interest, conventional isolation methods are frequently adapted from research protocols that depend on ultracentrifugation, density gradients, labor-intensive purification, or small-scale laboratory workflows. Such approaches are difficult to scale economically, may impose shear or processing burdens on vesicles, and are often poorly aligned with bulk ingredient production for consumer products.

A second challenge arises after isolation. Mammalian milk-derived vesicles can be sensitive to temperature fluctuation, dehydration stress, aggregation, and matrix incompatibility when introduced into real-world formulations. Methods suitable for laboratory characterization are not necessarily suitable for generating stable, formulation-ready ingredients that can be shipped, stored, and incorporated into serums, powders, capsules, or beverages.

Plant-derived vesicle or extract-based systems have been marketed in cosmetic settings, but these systems are materially and functionally distinct from mammalian milk-derived vesicles. Plant systems are often sold as extracts or vesicle-like preparations and do not necessarily present the same membrane preservation, lipid interference, or formulation challenges associated with mammalian extracellular vesicles.

Accordingly, there remains a need for a commercially practical process for obtaining mammalian milk-derived extracellular vesicles in useful quantities, preserving the vesicles and their associated molecular cargo, and placing the resulting materials into cosmetic and nutraceutical products without reliance on expensive and slow ultracentrifugation workflows.

### TECHNICAL DISTINCTION FROM PLANT-DERIVED SYSTEMS

Mammalian milk-derived extracellular vesicles present unique structural and processing challenges relative to plant-derived vesicle or extract-based systems. In particular, mammalian vesicles comprise lipid bilayer membranes and associated bioactive cargo that may be susceptible to disruption, aggregation, coalescence, or degradation during uncontrolled processing, temperature shifts, or dehydration.

In contrast, many plant-derived products marketed as exosomes are not isolated mammalian-type extracellular vesicles and do not require the same degree of membrane preservation or process discipline. The present invention recognizes that a commercially useful mammalian system must account for fat removal, clogging behavior, cold handling, stabilization chemistry, and formulation compatibility in a way that prior plant-oriented systems do not address.

By integrating staged filtration, optional tangential flow concentration, cryoprotective stabilization, and drying into a single commercially oriented workflow, the invention creates a practical route to formulation-ready mammalian milk-derived extracellular vesicle compositions.

### SUMMARY OF THE INVENTION

In one aspect, the invention provides methods for isolating extracellular vesicles from mammalian milk using sequential filtration through progressively smaller pore sizes without requiring ultracentrifugation. In some embodiments the method further includes optional tangential flow filtration to concentrate vesicles or remove smaller molecular components.

In another aspect, the invention provides stabilized extracellular vesicle compositions in liquid or dried form. Stabilization may be achieved with trehalose, milk-derived solids, sugars, polyols, proteins, or combinations thereof. The stabilized material may be dried by lyophilization, spray drying, vacuum drying, or analogous low-damage processes.

In another aspect, the invention provides formulation-ready compositions containing milk-derived extracellular vesicles for topical and ingestible use. Representative product categories include facial serums, creams, gels, hair serums, shampoos, conditioners, body care products, capsules, powders, beverages, protein blends, and creatine-containing nutraceuticals.

In another aspect, the invention provides scalable systems and methods for producing mammalian milk-derived extracellular vesicle ingredients at batch or continuous manufacturing scale, thereby enabling commercial supply, licensing, and ingredient platform applications.

### DEFINITIONS AND CLAIM SCOPE

As used herein, the terms “comprising,” “including,” and “having” are intended to be open-ended and mean that additional elements, steps, ingredients, or features may be present. The term “approximately” refers to a value within a practical manufacturing tolerance around the stated value, including tolerances of plus or minus 20% unless context suggests otherwise.

The term “extracellular vesicle” includes exosomes and related membrane-bound vesicles derived from mammalian milk, whether characterized by size, composition, origin, biological activity, or combinations thereof. The term “milk-derived” includes vesicles obtained from mammalian milk directly or from fractions, concentrates, or filtered derivatives of mammalian milk.

The term “formulation-ready” refers to a material that can be incorporated into a topical, ingestible, or related product with commercially practical handling and acceptable stability under intended storage and use conditions.

### DETAILED DESCRIPTION OF THE INVENTION

### 1. Source Material

In certain embodiments, the starting material comprises mammalian milk selected from bovine, caprine, ovine, human, buffalo, or other mammalian sources. Bovine milk is particularly useful due to availability, volume, and processing familiarity, but the invention is not limited thereto.

The milk may be raw, low-temperature pasteurized, vat-pasteurized, gently pasteurized, or otherwise processed prior to entry into the claimed workflow. The milk may be skim, whole, reduced-fat, partially defatted, or otherwise modified. In some embodiments low-temperature pasteurized skim or reduced-fat bovine milk is preferred due to reduced fat load and ease of filtration.

In some embodiments the source material is selected for low homogenization stress, low microbial burden, high consistency, or combinations thereof. Pre-processing may include refrigeration, settling, decanting, skimming, or removal of gross particulate matter before staged filtration begins.

### 2. Filtration-Based Isolation

In representative embodiments, extracellular vesicles are isolated using staged filtration through progressively decreasing pore sizes. A typical sequence may include a coarse filtration stage such as paper or coffee-filter-like media, followed by intermediate filtration such as approximately 20 μm, optionally followed by approximately 10 μm, approximately 5 μm, or approximately 2.5 μm filtration, and then fine filtration such as approximately 1 μm, approximately 0.45 μm, and optionally approximately 0.22 μm or smaller.

The sequence may be selected to reduce fat, cellular debris, and clogging while preserving vesicle recovery. In some embodiments, a cascade approach is used specifically to reduce filter fouling by avoiding immediate exposure of fine membranes to high fat and particulate loads. Filters may be arranged linearly, repeated, branched, or partially bypassed depending on input characteristics.

Filtration may be performed by gravity, vacuum assistance, gentle pressure, pump-assisted flow, dead-end flow, cross-flow, or combinations thereof. In some embodiments, Buchner funnels, vacuum flasks, filter holders, cartridge housings, or industrial housings are used. Filtration media may include polyethersulfone, polyvinylidene fluoride, nitrocellulose, cellulose acetate, polypropylene, polyester, paper, composite membranes, or combinations thereof.

In some embodiments, membrane wetting or pre-conditioning steps are used to improve throughput or reduce bubble formation. In other embodiments, filtration is repeated at the same pore size until a desired clarity, lipid reduction, or vesicle enrichment condition is achieved. Filtration order, pore sizes, and membrane materials may be varied without departing from the scope of the invention.

### 3. Temperature-Controlled Processing

In some embodiments, one or more stages of the process are performed under reduced temperature conditions to preserve vesicle integrity and reduce degradation or aggregation. Representative temperatures include approximately 0 °C to 10 °C, approximately 1 °C to 8 °C, or chilled conditions approximating refrigerated processing.

Temperature control may be achieved by refrigerated rooms, ice baths, chilled vessels, jacketed systems, cooled transfer lines, or intermittent cold-hold steps. In laboratory embodiments, Buchner flasks or receiving vessels may be maintained in ice baths during filtration. In scaled embodiments, process tanks and lines may be jacket-cooled or otherwise temperature controlled.

Temperature control may be continuous throughout processing or selectively applied during critical steps including prefiltration, fine filtration, concentration, stabilization, or reconstitution.

### 4. Tangential Flow Filtration

In certain embodiments, tangential flow filtration (TFF) is used as an alternative or additional concentration step. TFF may be applied before, after, or between one or more staged filtration steps. The purpose of TFF may include volume reduction, removal of smaller soluble components, enrichment of vesicle-containing fractions, buffer exchange, or combinations thereof.

TFF modules may include hollow fiber, flat sheet, cassette, spiral wound, or equivalent geometries. Membrane cutoffs may range from approximately 100 kDa to approximately 1,000 kDa, including approximately 300 kDa, 500 kDa, 750 kDa, or other values suitable for retaining extracellular vesicles while allowing passage of smaller contaminants or soluble components.

In some embodiments, TFF is optional and not required for infringement. In other embodiments, TFF provides a scalable concentration route that avoids ultracentrifugation and improves commercial throughput. TFF may be performed at one or more concentration factors, including approximately 2×, 4×, 10×, or more, depending on desired yield and formulation goals.

### 5. Stabilization Systems

Following isolation or concentration, extracellular vesicles may be combined with a stabilization composition to preserve membrane integrity and associated molecular cargo during drying, storage, shipping, and formulation. Stabilizers may include trehalose, sucrose, fructose, erythritol, glycerin, diglycerol, milk-derived solids, proteins, peptides, salts, buffers, or combinations thereof.

In representative embodiments, trehalose and milk-derived solids are used together to form a protective amorphous matrix during drying. Example working amounts include approximately 30-80 g/L trehalose and approximately 10-30 g/L milk-derived solids, including about 65 g trehalose and about 21 g milk-derived solids per 1800 mL of starting material or a scaled equivalent thereof.

The stabilizer system may be selected to reduce membrane rupture, collapse, fusion, phase separation, microRNA degradation, or loss of dispersibility. Stabilizers may be mixed before freezing, before concentration, after concentration, during reconstitution, or in multiple stages.

### 6. Drying Methods

The invention contemplates multiple drying strategies. In one embodiment, the stabilized extracellular vesicle suspension is lyophilized. The material may be pre-frozen for approximately 6-48 hours, including approximately 24 hours, before entry into a freeze dryer. Drying may occur under reduced pressure, such as approximately 50-500 mTorr or approximately 100-200 mTorr, with or without heated shelves.

In some embodiments, trays are frozen prior to loading into the freeze dryer. In some embodiments, shelf heat is minimized or turned off to reduce thermal stress. Freeze-drying cycles may be adjusted to obtain a free-flowing or friable dried cake suitable for post-processing into powder.

In other embodiments, spray drying or vacuum drying is used, optionally with protective matrices designed to preserve vesicle integrity. Drying parameters may be chosen to balance throughput, stability, residual moisture, and downstream dispersibility.

### 7. Post-Processing and Storage

After drying, the material may be mechanically reduced in size, milled, crushed, broken, or sieved into desired particle ranges. Representative mesh ranges include approximately 20-100 mesh, including approximately 40 and 60 mesh in sequence.

The processed material may be vacuum sealed, packaged under reduced oxygen conditions, sealed in barrier pouches, sealed in mylar, stored under refrigerated conditions, or stored frozen. Multiple packaging layers may be used. In some embodiments a vacuum-sealed bag is placed within a secondary barrier package to improve stability.

The dried material may be later reconstituted into liquids, dispersed directly into a formulation phase, or blended into dry mixes without full reconstitution.

### 8. MicroRNA Functional Positioning

In certain embodiments, the extracellular vesicles comprise distinct microRNA profiles characteristic of mammalian milk-derived vesicles. Such microRNA content may include, without limitation, miR-148a, miR-144, miR-30d, miR-146b, and additional microRNAs naturally present in mammalian milk-derived extracellular vesicles.

These microRNA profiles may differ from those of plant-derived systems and may contribute to biological activity relevant to skin barrier support, hydration, scalp condition, general physiological support, and other topical or ingestible uses. The invention is not limited to any single microRNA, exact abundance profile, or specific mechanism of action.

In addition to providing distinct microRNA-containing extracellular vesicles, the invention enables scalable concentration of such vesicles using filtration-based methods that avoid ultracentrifugation. The staged filtration and optional TFF processes described herein allow concentration of extracellular vesicles while preserving structural integrity and associated molecular cargo, including microRNAs.

While extracellular vesicles and associated microRNAs have been described in various biological contexts, prior methods have not been shown to enable the production of stable, formulation-ready compositions suitable for incorporation into cosmetic and nutraceutical products at commercial scale without reliance on ultracentrifugation. The present invention addresses this limitation by providing a practical and scalable system for concentrating, stabilizing, and delivering milk-derived extracellular vesicles containing biologically active microRNAs in both topical and ingestible formulations.

### 9. Mammalian Source Advantage

Mammalian-derived extracellular vesicles may be obtained from a variety of biological sources; however, many such sources present practical limitations for scalable processing, formulation compatibility, regulatory simplicity, routine sourcing, or consumer product integration. In contrast, milk-derived extracellular vesicles provide a readily available, processable, and scalable source of mammalian extracellular vesicles that can be isolated, stabilized, and incorporated into formulation-ready compositions.

The present invention leverages these advantages by enabling concentration and preservation of milk-derived extracellular vesicles in forms suitable for both topical and ingestible applications. This approach provides a practical pathway for delivering mammalian extracellular vesicle-associated molecular cargo, including microRNAs, in commercially viable products.

### 10. Formulation Systems

The extracellular vesicle compositions of the invention may be incorporated into topical formulations including serums, gels, creams, lotions, emulsions, sprays, masks, primers, hair serums, scalp products, shampoos, conditioners, body washes, soaps, ointments, and related cosmetic or personal care systems.

In representative serum embodiments, extracellular vesicles may be dispersed directly into one phase of a multi-component formulation. In certain embodiments, the formulation may further include water, glycerin, diglycerol, trehalose, betaine, niacinamide, fructose, erythritol, acrylate or silicone-containing film formers, sodium hyaluronate, nonfat dry milk, sunflower lecithin, allantoin, EDTA, preservatives, and humectants.

Extracellular vesicle loading may vary widely depending on intended product positioning. Representative loading levels include approximately 0.001% to 20% by weight, approximately 0.01% to 10% by weight, or approximately 0.1% to 5% by weight. In some serum embodiments, approximately 0.1 g to 10 g dried extracellular vesicle powder may be used per formulation unit, including approximately 2 g or 3 g per 30 mL bottle or scaled equivalents thereof.

The invention also contemplates nutraceutical compositions including capsules, sachets, powders, tablets, stick packs, beverages, gels, shots, protein mixes, and creatine-containing blends. The extracellular vesicles may be combined with proteins, creatine, amino acids, electrolytes, sweeteners, flavor systems, stabilizers, or other nutraceutical ingredients.

### 11. Functional Applications

Expanded applications include cosmetic, personal care, nutraceutical, and therapeutic-support categories. Representative uses include improving the appearance of skin, supporting hydration, supporting barrier function, supporting scalp condition, supporting hair appearance, supporting gut function, supporting general wellness, supporting healthy aging processes, and supporting hybrid topical-plus-ingestible product systems.

No statement herein requires limitation to any single mechanism of action. The invention encompasses compositions and methods that deliver mammalian milk-derived extracellular vesicles and associated cargo in commercially practical formats.

### 12. Representative Examples

Example 1—Filtration and Lyophilization Workflow. A low-temperature pasteurized bovine milk source is maintained under chilled conditions. The material is passed through a coarse filter, then through a 20 μm filter, then through a 2.5 μm filter, and then through a 0.45 μm filter. The filtered fraction is optionally subjected to 750 kDa TFF. Trehalose and milk-derived solids are added. The composition is pre-frozen for approximately 24 hours and lyophilized under reduced pressure. The dried cake is crushed, sieved, and vacuum packaged.

Example 2—High-Concentration Topical Serum. Dried extracellular vesicle powder prepared according to the invention is dispersed into a topical serum phase containing water, glycerin, diglycerol, trehalose, betaine, niacinamide, fructose, erythritol, film-forming polymer, sodium hyaluronate, sunflower lecithin, allantoin, and preservative components. A final loading of approximately 3 g extracellular vesicle powder per 30 mL bottle is obtained. The resulting serum provides a high-concentration product suitable for skin hydration and cosmetic use.

Example 3—Standard Topical Serum. A similar composition is prepared with approximately 2 g extracellular vesicle powder per 30 mL bottle to provide a standard concentration topical cosmetic embodiment.

Example 4—Nutraceutical Powder Blend. Dried extracellular vesicle powder is blended with protein and/or creatine in a dry nutraceutical composition suitable for oral consumption. The composition may additionally include flavors, sweeteners, electrolytes, or functional actives and may be packaged as a powder, stick pack, or beverage base.

Example 5—Hair Care Product. Dried or liquid extracellular vesicle compositions are incorporated into a hair serum, shampoo, or conditioner with a cosmetically acceptable carrier to support scalp condition and hair appearance.

### 13. Non-Limiting Embodiments and Equivalents

As used herein, all disclosed ranges include endpoints, subranges, and individual values therein. Features described in connection with one embodiment may be combined with features of another embodiment unless explicitly incompatible. The disclosed embodiments are illustrative and not limiting.

Any process that achieves isolation, concentration, stabilization, or formulation of extracellular vesicles derived from mammalian milk using filtration-based or other non-ultracentrifugation methods in a substantially equivalent manner may fall within the scope of the present invention.

What is claimed is:

1. A method of isolating extracellular vesicles from mammalian milk comprising sequential filtration through progressively decreasing pore sizes, wherein the method is performed without ultracentrifugation.

2. The method of claim 1, wherein the filtration comprises at least two filtration steps selected from pore sizes greater than 10 μm, approximately 2.5 μm, and approximately 0.45 μm.

3. The method of claim 1, wherein the filtration is performed under temperature-controlled conditions.

4. The method of claim 3, wherein the temperature is maintained between approximately 0 °C and 10 °C.

5. The method of claim 1, further comprising removal of lipid fractions prior to filtration.

6. The method of claim 1, wherein filtration is performed using membranes selected from polyethersulfone (PES), polyvinylidene fluoride (PVDF), nitrocellulose, or combinations thereof.

7. The method of claim 1, further comprising concentrating extracellular vesicles using tangential flow filtration.

8. The method of claim 7, wherein tangential flow filtration utilizes membranes with a molecular weight cutoff between approximately 100 kDa and 1,000 kDa.

9. The method of claim 8, wherein the membrane cutoff is approximately 750 kDa.

10. The method of claim 1, wherein concentration is achieved through repeated filtration cycles.

11. The method of claim 1, wherein the method is scalable for batch or continuous production.

12. The method of claim 1, further comprising combining extracellular vesicles with a stabilizing composition.

13. The method of claim 12, wherein the stabilizing composition comprises trehalose.

14. The method of claim 12, wherein the stabilizing composition further comprises milk-derived solids.

15. The method of claim 12, wherein the stabilizing composition comprises one or more sugars selected from trehalose, sucrose, fructose, or erythritol.

16. The method of claim 12, wherein the stabilizing composition forms an amorphous matrix.

17. The method of claim 1, further comprising drying the extracellular vesicles.

18. The method of claim 17, wherein drying comprises lyophilization.

19. The method of claim 18, wherein lyophilization includes pre-freezing for at least 12 hours.

20. The method of claim 17, wherein drying comprises spray drying.

21. The method of claim 17, further comprising producing a powder form of extracellular vesicles.

22. A composition comprising extracellular vesicles derived from mammalian milk produced by the method of claim 1.

23. The composition of claim 22 in dried powder form.

24. The composition of claim 22 in liquid suspension form.

25. The composition of claim 22, wherein the extracellular vesicles comprise microRNAs including miR-148a, miR-144, miR-30d, and miR-146b.

26. The composition of claim 22, wherein extracellular vesicles are present at a concentration between approximately 0.001% and 20% by weight.

27. A topical formulation comprising the composition of claim 22 and a cosmetically acceptable carrier.

28. The formulation of claim 27, wherein the formulation is a serum.

29. The formulation of claim 27, wherein the formulation further comprises niacinamide.

30. The formulation of claim 27, wherein the formulation further comprises hyaluronic acid.

31. The formulation of claim 27, wherein the formulation further comprises betaine.

32. The formulation of claim 27, wherein the formulation is configured to improve skin hydration or appearance.

33. The formulation of claim 27, wherein the formulation is a hair care composition.

34. The formulation of claim 33, wherein the composition is configured to support scalp condition.

35. The formulation of claim 33, wherein the composition is configured to improve hair appearance.

36. A nutraceutical composition comprising extracellular vesicles derived from mammalian milk.

37. The composition of claim 36 in capsule form.

38. The composition of claim 36 in powder form.

39. The composition of claim 36 in beverage form.

40. The composition of claim 36 further comprising protein.

41. The composition of claim 36 further comprising creatine.

42. The composition of claim 36 configured to support hydration, gut function, or general wellness.

43. A method of improving skin appearance comprising applying the formulation of claim 27.

44. A method of supporting hydration comprising administering the composition of claim 36.

45. A method of supporting gut health comprising administering the composition of claim 36.

46. A method of supporting systemic wellness comprising administering the composition of claim 36.

47. A system comprising: (a) a filtration-based extracellular vesicle isolation process; (b) a stabilization composition; and (c) a formulation system configured to produce a topical or ingestible product.

48. The system of claim 47, wherein the system operates without ultracentrifugation.

49. The system of claim 47 configured for commercial-scale production.

50. The method of claim 1, wherein the absence of ultracentrifugation is replaced by any filtration-based concentration process.

51. The composition of claim 22 wherein extracellular vesicles are derived from any mammalian milk source.

52. The formulation of claim 27 wherein the extracellular vesicles are incorporated into any cosmetically acceptable delivery system.

53. A composition comprising extracellular vesicles derived from mammalian milk, wherein the vesicles are produced using a process that does not include ultracentrifugation.

54. A method of producing a commercially scalable extracellular vesicle composition from mammalian milk comprising filtration-based isolation, stabilization, and formulation into a product selected from topical and ingestible formats.
